(19) United States
(12) Patent Application Publication
(10) Pub. No.: US 2008/0161843 A1
(43) Pub. Date: Jul. 3, 2008

(54) Vessel support device and method of vessel harvesting

(76) Inventors: Cynthia T. Clague, Minnetonka, MN (US); Michael J. Hobday, Lino Lakes, MN (US); Raymond W. Usher, Coon Rapids, MN (US); Roderick E. Briscoe, Rogers, MN (US); Katherine S. Olig, Eden Prairie, MN (US); Ana R. Menk, Minneapolis, MN (US); Christopher P. Olig, Eden Prairie, MN (US); Eric A. Meyer, Andover, MN (US); Steven C. Christian, New Brighton, MN (US); Thomas P. Daigle, Corcoran, MN (US); Robert H. Reetz, Rockford, MN (US); Jeffrey D. Sandstrom, Forest Lake, MN (US); James R. Keogh, Maplewood, MN (US); Matthew D. Bonner, Plymouth, MN (US); Scott E. Jahns, Hudson, WI (US); Philip J. Haarstad, Chanhassen, MN (US)

Correspondence Address: MEDTRONIC, INC., 710 MEDTRONIC PARKWAY NE, MINNEAPOLIS, MN, 55432-9924, US

(21) Appl. No.: 11/974,922

(22) Filed: Oct. 16, 2007

Related U.S. Application Data
(60) Provisional application No. 60/852,020, filed on Oct. 16, 2006.

(51) Int. Cl. A61B 17/32; A61M 5/00; A61M 31/00; A61M 5/178; A61B 19/00

(52) U.S. Cl. 606/167; 604/264; 604/509; 604/164.13; 128/898

(57) ABSTRACT

Embodiments of the invention provide a vessel support system and a method of vessel harvesting. The system can include a cutting device, a catheter adapted to be inserted into a section of the vessel in order to support the vessel as the cutting device is advanced over the vessel, and a cannula adapted to be coupled to the vessel and adapted to receive the catheter as the catheter is inserted into the section of the vessel. The method can include orienting a cutting device coaxially with the cannula and the catheter and advancing the cutting device over the cannula, the catheter, and the section of the vessel in order to core out the section of the vessel and a portion of the surrounding tissue.

### RELATED APPLICATIONS

This application claims priority under 35 U.S.C. §119 to U.S. Provisional Patent Application Ser. No. 60/852,020, filed on Oct. 16, 2006, the entire contents of which is hereby incorporated by reference.

### FIELD OF THE INVENTION

This invention relates generally to biomedical systems and methods. More specifically, the invention relates to systems and methods for harvesting a vessel section.

### BACKGROUND

Heart disease, specifically coronary artery disease, is a major cause of death, disability, and healthcare expense in the United States and other industrialized countries. A common form of heart disease is atherosclerosis, in which the vessels leading to the heart are damaged or obstructed by plaques containing cholesterol, lipoid material, lipophages, and other materials. When severely damaged or obstructed, one or more of the vessels can be bypassed during a coronary artery bypass graft (CABG) procedure. CABG surgery is performed about 350,000 times annually in the United States, making it one of the most commonly performed major operations.

To prevent rejection, the graft material is preferably a blood vessel harvested from elsewhere within a patient's body. The most frequently used bypass vessel is the saphenous vein from the leg. Because the venous system of the leg is redundant, other veins that remain within the patient's leg are able to provide return blood flow following removal of the saphenous vein.

Various methods have been used to harvest the saphenous vein. Until recently, the typical procedure involved making a single long incision that overlies the entire length of the vein, extending from a patient's groin to at least the knee and often to the ankle. This method results in substantial postoperative pain, with patients frequently complaining more of discomfort at the site of the leg vein harvesting than of pain from their CABG surgery wound. In addition, such an extensive incision site is subject to infection and delayed healing, especially in patients with poor circulation, which not infrequently accompanies coronary artery disease. The disfiguring scar from such a large incision is also of concern to some patients.

Less invasive procedures are preferred, and surgical devices and techniques now exist that allow the saphenous vein to be harvested through one or more small, transverse incisions along the length of the vein, generally using an endoscope. Endoscopic procedures yield reduced wound complications and superior cosmetic results compared with traditional methods of vein harvesting. However, this procedure requires considerable manipulation of the vein, has a high conversion rate when visualization is obscured by bleeding or the procedure is taking too long and often requires stitches to repair the vein following harvest. Further, it is generally tedious, time consuming, and relatively complex, requiring extensive accessory equipment and a substantial learning curve for the surgeon.

### SUMMARY

Some embodiments of the invention provide a system for harvesting a section of a vessel from surrounding tissue. The system can include a cutting device adapted to surround the vessel along the section of the vessel and adapted to be moved along the section of the vessel in order to cut the tissue around the vessel. The system can also include a catheter adapted to be inserted into the section of the vessel in order to support the vessel as the cutting device is advanced over the vessel. The system can further include a cannula adapted to be coupled to the vessel and adapted to receive the catheter as the catheter is inserted into the section of the vessel.

According to a method of the invention, a section of a vessel can be harvested from surrounding tissue by making a first incision at a proximal end of the section of the vessel, and making a second incision at a distal end of the section of the vessel. The method can include inserting a cannula into the proximal end of the vessel, and securing the proximal end of the vessel to the cannula. The method can also include inserting a catheter through the cannula and into the section of the vessel, and orienting a cutting device coaxially with the cannula and the catheter. The method can further include advancing the cutting device over the cannula, the catheter, and the section of the vessel in order to core out the section of the vessel and a portion of the surrounding tissue.

One embodiment of the invention provides an intravascular balloon catheter for use in supporting a section of a vessel being harvested from surrounding tissue with a cutting device. The catheter includes a balloon with a proximal end and a distal end, the proximal end being plugged and the distal end including a routing neck. The balloon is adapted to be inflated in the vessel in order to support the vessel as the cutting device is advanced along the vessel. The catheter also includes a stylet coupled to the routing neck of the balloon. The stylet includes a flexible tip and a coiled wire adapted to navigate through the vessel in order to position the balloon in the section of the vessel.

Another embodiment of the invention provides a cannula for use in harvesting a section of a vessel. The cannula includes a distal tip adapted to be inserted into and secured to a proximal end of the section of the vessel. The cannula also includes a valve adapted to prevent fluid flow out of the proximal end of the section of the vessel, with the valve positioned in a proximal end of the cannula. The cannula further includes a tension-coupling member adapted to be coupled to a tensioning device, with the tension-coupling member being coupled to the proximal end of the cannula. The tension-coupling member includes at least one groove adapted to receive at least one raised bump of a tensioning device member adapted to be coupled to a tensioning device.

### DESCRIPTION OF THE DRAWINGS

FIG. 1 is an illustration of a system for harvesting a vessel section in accordance with embodiments of the invention;

FIGS. 2A-2B are flow diagrams of vessel harvesting methods in accordance with embodiments of the invention;

FIGS. 3A-3D are illustrations of a roll-out intravascular sheath for harvesting a vessel section in some embodiments of the invention;

FIGS. 4A-4F are illustrations of a one piece intravascular catheter balloon and stylet for harvesting a vessel section in some embodiments of the invention;

FIGS. 5A-5E are illustrations of a cannula and tensioning device member for use in harvesting a vessel section in some embodiments of the invention;

FIG. 6 is an illustration of an insertion device for a flow delivered tethered balloon for use in harvesting a vessel section in some embodiments of the invention;

FIGS. 7A-7C are illustrations of vessel support devices for use in harvesting a vessel section in some embodiments of the invention;

FIG. 8 is an illustration of a vein illumination device for use in harvesting vessel sections in some embodiments of the invention;

FIG. 9 is an illustration of a catheter guide for use in harvesting vessel sections in some embodiments of the invention;

FIG. 10 is an illustration of a hemostatic control method for use in harvesting vessel sections in some embodiments of the invention; and

FIG. 11 is an illustration of a vessel location and hemostasis method for use in harvesting vessel sections in some embodiments of the invention.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

The following discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The following detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

As used in this specification and in the appended claims, the terms “distal” and “proximal” are with reference to the operator when the device is in use.

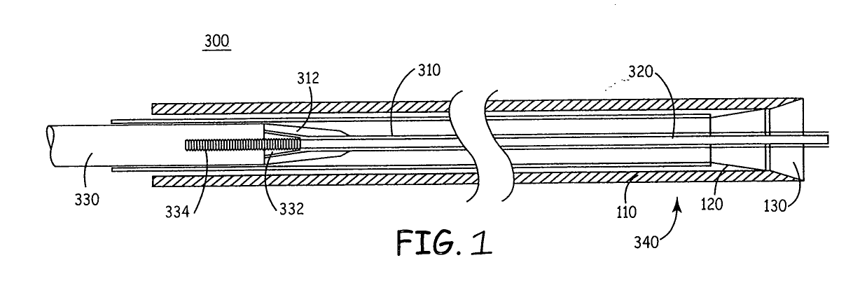

FIG. 1 illustrates a vessel harvesting system 300 according to some embodiments of the invention. The system 300 can include a catheter 310, a rod 320, a handle 330, and a tubular cutting device 340. The system 300 can also include a guidewire and can be used in conjunction with a hemostatic control method for treating severed branch vessels.

The catheter 310 and the guidewire can be constructed of a suitable biocompatible materials or combinations thereof, for example, a polymer, stainless steel, nitinol, composites, etc. The lengths of the catheter 310 and guidewire can be roughly determined by the length of the vessel section to be harvested. The rod 320, the catheter 310, and/or the guidewire can be coated with a lubricious, slippery material. For example, the catheter 310 can be coated with a slippery material to decrease friction between the catheter 310 and the vessel to ease passage of the catheter 310 into the vessel and decrease the possibility of damaging the vessel interior. The coating can be, for example, a hydrogel coating, polyacrylamide, polyethylene oxide, Teflon, parylene, etc. The coating can also contain one or more biological agents, such as an anticoagulant or an antithrombogenic agent to reduce clotting inside the vessel during the harvest procedure. In one embodiment, the anticoagulant can be heparin.

In some embodiments, the coating can contain one or more vasoactive agents or drugs, such as vasodilative agents or drugs and/or vasoconstrictive agents or drugs. Examples of a vasodilative drugs include, but are not limited to, a vasodilator, an organic nitrate, isosorbide mononitrate, a mononitrate, isosorbide dinitrate, a dinitrate, nitroglycerin, a trinitrate, minoxidil, sodium nitroprusside, hydralazine hydrochloride, nitric oxide, nicardipine hydrochloride, fenoldopam mesylate, diazoxide, enalaprilat, epoprostenol sodium, a prostaglandin, milrinone lactate, a bipyridine and a dopamine D₁-like receptor agonist, stimulant or activator. Examples of vasoconstrictive drugs include, but are not limited to, a vasoconstrictor, a sympathomimetic, methoxamine hydrochloride, epinephrine, midodrine hydrochloride, desglymidodrine, and an alpha-receptor agonist, stimulant or activator. In one embodiment, vasoactive agents or drugs can be administered via one or more bolus injections and/or infusions or combinations thereof. The injections and/or infusions can be continuous or intermittent. The injections and/or infusions can be made directly into the vessel section to be harvested.

In one embodiment, the catheter 310 is strong enough to receive the rod 320 within a lumen of the catheter 310 and has an outer diameter smaller than the narrowest inner diameter of the vessel to be harvested. The catheter 310 can include one or more lumens. In one embodiment, the catheter 310 can include one or more fluid openings fluidly connected to one or more lumens for delivering or introducing fluids into one or more portions of the vessel to be harvested. The one or more lumens can be fluidly coupled to one or more fluid sources. For example, one or more fluids can be introduced from one or more fluid sources into the vessel to be harvested through the one or more fluid openings prior to removing the catheter 310 from the harvested the vessel. One or more fluids also can be introduced into the vessel through the one or more fluid openings while introducing the catheter 310 into the vessel to be harvested. In one embodiment, suction or a negative pressure can be introduced into the vessel through the one or more fluid openings. For example, suction can be provided from a suction source coupled to the one or more lumens which, in-turn, are coupled to the one or more fluid openings to draw and hold the vessel to be harvested to the catheter 310 while advancing the cutting device 340 over the vessel and along the catheter 310.

In one embodiment, the catheter 310 can include one or more balloons, distensible members and/or inflatable members fluidly coupled to one or more lumens. Following placement of the catheter 310 into the vessel section to be harvested, one or more inflatable members can be inflated via a gas or liquid, thereby securing the vessel to the catheter 310. The gas or liquid can be, for example, air, carbon dioxide, or saline. The one or more inflatable members can be inflated while advancing the cutting device 340 over the vessel and along the catheter 310.

In some embodiments, a balloon catheter 310 that provides vessel support can also provide a centering function. The balloon catheter 310 can include one or more inflatable structures or elements that can be alternately inflated and deflated. The inflatable structure or structures can expand into the lumen of an inner or outer tubular member of the cutting device 340. The expansion can force the vessel and the tissue surrounding it into the center of the member to thereby center the cutting element 340 on the vessel. The structure or structures can be inflated to center the vessel and then the cutting element 340 used to cut the tissue adjoining the vessel. The structure or structures can then be deflated to advance the cutting device 340 along the vessel. After advancing the cutting device 340, the structure or structures can again be inflated and the cutting element 340 can be used to cut the tissue around the vessel. The process of incrementally inflating, cutting, deflating, and advancing can be repeated until the entire section has been excised. In one embodiment, the structure or structures can be inflated the entire time the cutting element 340 is advanced along the vessel.

The rod 320 can be an appropriate rigid biocompatible material, for example stainless steel or a rigid polymer. In one embodiment, the rod 320 is long enough to extend beyond at least the proximal end of the vessel section to be harvested and to be attached to the handle 330.

The handle 330 can be constructed of stainless steel; however, other appropriate materials such as other metals and/or suitable polymers can be used. A proximal end of the catheter 310 can be removably attached to the handle 330. FIG. 1 illustrates a taper fitting 312 on the proximal end of the catheter 310 that slips over a complementary taper fitting 332 on the distal end of the handle 330 and secures the catheter 310 to the handle 330. Other fittings, for example, a screw fitting, can also be used. In an alternative embodiment, a proximal portion of the catheter 310 can instead be attached to a proximal portion of the rod 320 after the rod 320 has been inserted into the catheter 310. The catheter 310 can also attach to the proximal or mid-portion of the handle 330 and the vessel can attach to the distal end of the handle 330.

The handle 330 can include a cavity 334 within which a proximal portion of the rod 320 is received. The cavity 334 can be contained within the handle 330, as shown in FIG. 1. Alternatively, the cavity 334 can extend through the handle 330, allowing the length of the portion of the rod 320 that extends from the handle 330 to be variable. A setscrew or other appropriate device can be used to secure the rod 320 within the cavity 334.

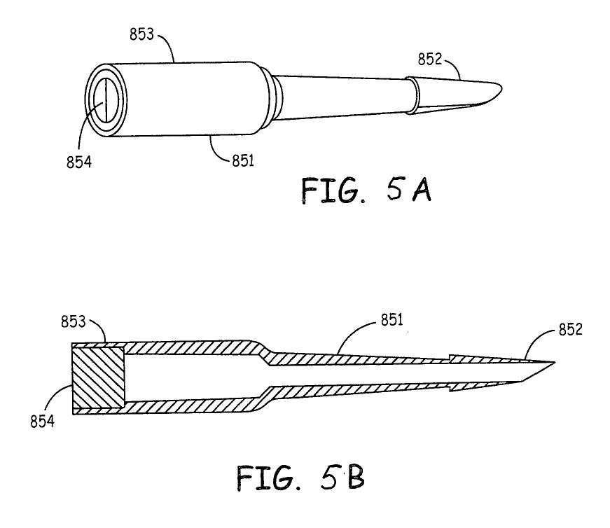

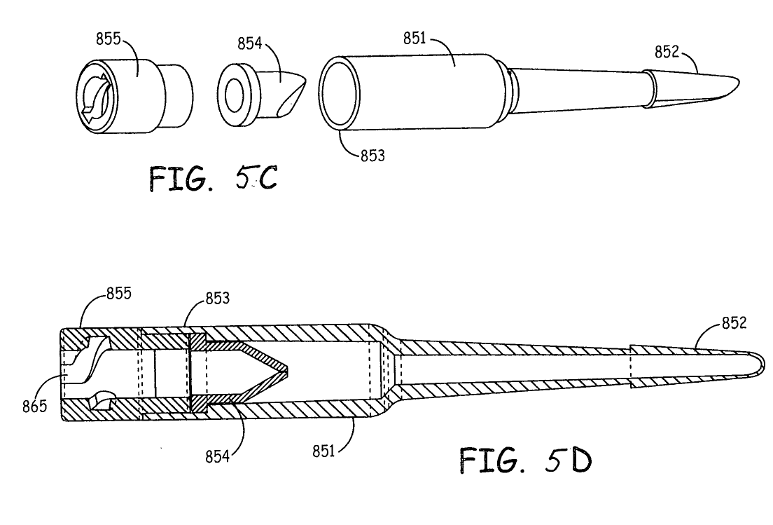

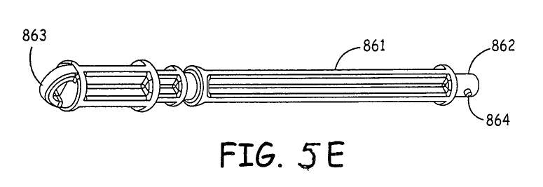

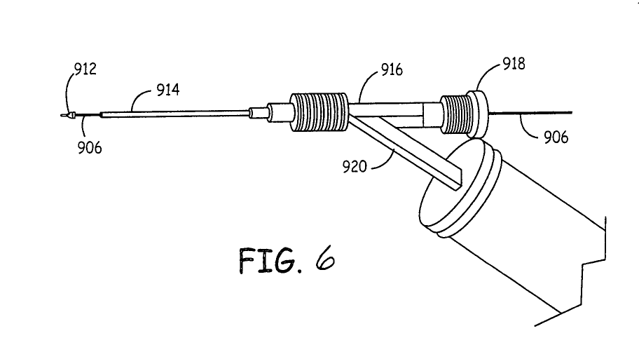

Alternatively, a vessel cannula 851 (as shown and described with respect to FIGS. 5A-5D) can be secured to the vessel. The catheter 310 can be passed through the cannula 851 into the vessel until a small portion remains within the cannula 851. The catheter 310 can then be inflated or expanded to support the vessel. The expansion in the cannula 851 can help to hold the catheter 310 in place. A tensioning device can then be attached to the cannula 851 to hold the end of the vessel in place while the cutting device 340 is advanced along the outside of the vessel.

As shown in FIG. 1, the cutting device 340 can include an outer tubular member 110 and an inner tubular member 120. The outer tubular member 110 can include a cutting element 130 positioned adjacent to its distal end. In some embodiments, the tubular members 110, 120 can be advanced independently of each other. The cutting device 340 can include a centering member for centering the vessel within the cutting device 340. In an alternative embodiment, the cutting device 340 can include a single tubular member 110 having a cutting element 130 positioned adjacent to its distal end.

In some embodiments, the cutting device 340 slides over the handle 330. An inner lumen of the cutting device 340 provides a close-sliding fit for the handle 330. As shown in FIG. 1, the handle 330 extends beyond a proximal end of the cutting device 340, thereby enabling an operator of the system 300 to grasp a proximal portion of the handle 330 while advancing the cutting device 340 over the distal portion of the handle 330 and over the vessel section to core out the vessel section and tissue adjoining the vessel section. Only a distal portion of handle 330 is shown in FIG. 1.

With the vessel harvesting system 300, a hemostatic control method can be used to treat branch vessels severed by the cutting device 340 as it is advanced over the vessel section. Various hemostatic control methods are possible. For example, the hemostatic control method can include the use of a biological sealant or tissue adhesive, for example a platelet gel that is prepared from the patient's blood and injected or otherwise introduced along the track of the cutting device 340. Alternatively, or in combination with a biological sealant, a biocompatible or biodegradable tube can be enclosed within the cutting device 340 to be delivered as the cutting device 340 is advanced over the vessel or after the cutting device 340 has completed coring out the vessel and adjoining tissue. A hemostatic control tube can exert pressure on the cut branch vessels and can be either removed or, in the case of a biodegradable tube, left in place to dissolve or degrade over a period of a few days, for example. Alternatively, the exterior of the tubular cutting device 340 can be coated with or deliver a procoagulant material such as thrombin, collagen, a thrombotic polymer, or activating agent such as kaolin or celite to promote clotting of the tissues as the cutting device 340 is harvesting the vessel or after harvesting the vessel. The tubular cutting device 340 can provide a hemostatic control method as it exerts pressure on the cut branch vessels while it remains within the patient's body. A fluid or gas, e.g. saline or carbon dioxide, can be supplied at the tip of the tool to deliver the fluid or gas into the tissue in the region where the vessel is being harvested. The supplied fluid or gas will accumulate and increase the pressure around the vessel being harvested. The increased pressure can exceed the pressure in the severed vessel branches and provide some hemostatic control by collapsing the vessels and preventing blood from exiting the severed end. A drain can be inserted at the end of the harvesting procedure to deal with any bleeding that does occur.

An alternative embodiment of the vessel harvesting system can include a rod 320, a handle 330 attached to the rod 320, and a tubular cutting device 340. This system is similar to system 300 described above, but does not include a catheter 310. Rather, the rod 320 is inserted directly into the vessel.

Yet another embodiment of the vessel harvesting system can include a catheter 310, a rod 320, and a tubular cutting device 340. Again, this system is similar to system 300, with the exception that no handle 330 is included in this system. Instead of advancing over a handle 330, the cutting device 340 can be oriented coaxial with the rod 320. The rod 320, when fully inserted into the catheter 310 within the vessel to be harvested, can extend far enough outside of the vessel to allow the cutting device 340 to be aligned over the rod 320. The catheter 310 can be attached to the rod 320 before advancing the cutting device 340 over the rod 320, the catheter 310, and the vessel to core out the vessel section and the tissue adjoining the vessel section.

Another embodiment of the system can include a rod or guidewire 320 that extends beyond the distal end of the vessel and beyond the proximal end of the handle 330. The portion of the rod or guidewire 320 that extends beyond the vessel to be excised and the cutting device 340 can be used to anchor the rod or guidewire 320 to a stable object, such as a surgical table or a bedrail. An anchor device can be used to hold the rod or guidewire 320 and a support device can be used to raise or lower the rod or guidewire 320 to a height necessary to be level with the vessel being excised. The anchor and support devices can hold the rod or guidewire 320 steady, straight, and level for the cutting device 340 to follow. In one embodiment, the vessel can be attached to the catheter 310 and the rod and/or the guidewire 320. In one embodiment, the catheter 310 and the rod or guidewire 320 can be coupled to a tensioning device.

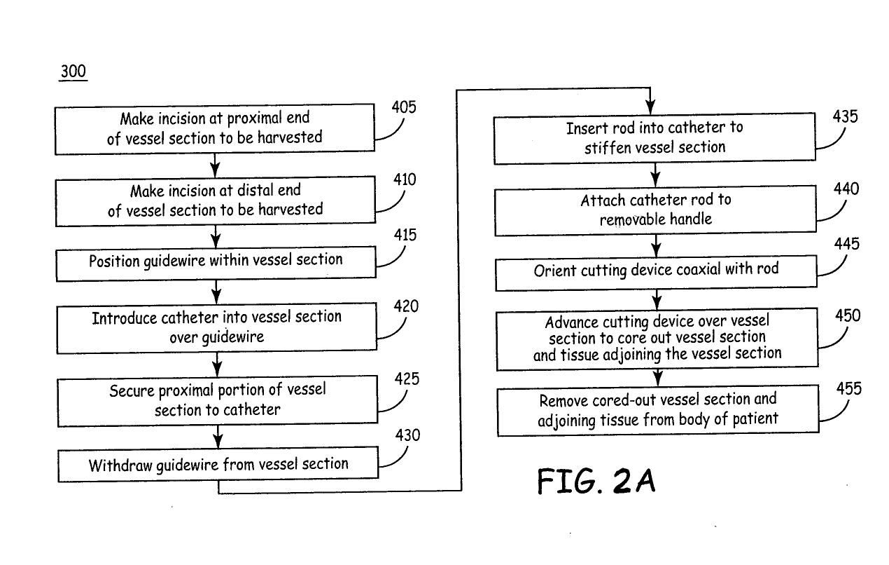

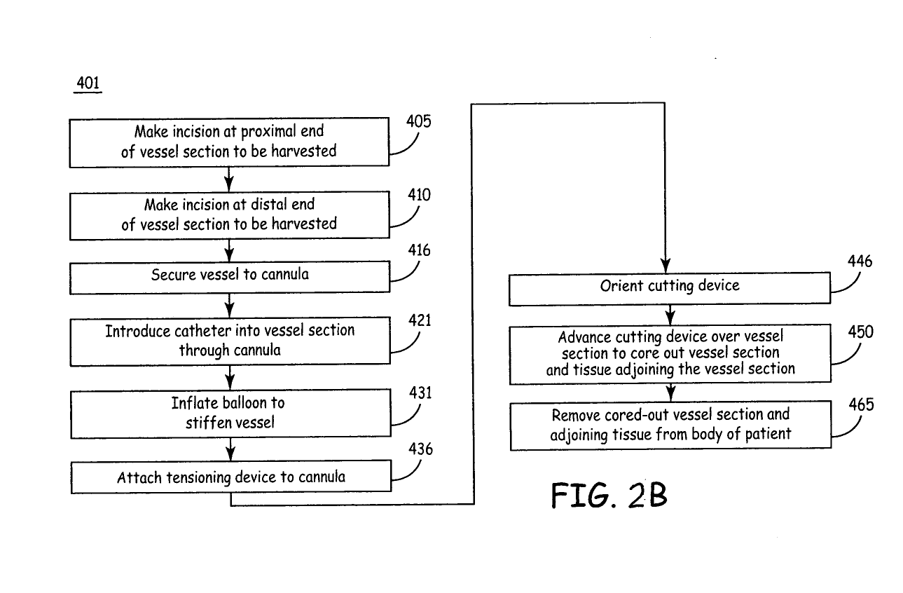

FIG. 2A is flow diagram of a vessel harvesting method according to one embodiment of the invention. In this embodiment, a first incision is made at a point corresponding to a proximal end of the vessel section to be harvested (Block 405). A second incision is made at a point corresponding to a distal end of the vessel section (Block 410). A guidewire is then positioned within the vessel section (Block 415). Alternatively, the guidewire can be inserted into the vessel before the second incision is made. Inserting the guidewire prior to making the second incision can aid in determining the optimal location for the second incision. Once the second incision has been made, the guidewire is positioned such that it extends beyond and outside of the vessel section at both the distal and proximal ends of the section.

A catheter is introduced into the vessel section over the previously placed guidewire (Block 420). A proximal portion of the vessel section is secured to the catheter (Block 425), for example by suturing the vessel onto a barb positioned adjacent to the proximal end of the catheter. Alternatively, the catheter can be introduced into the vessel without a guidewire being previously placed.

The guidewire (if present) is withdrawn (Block 430), and a rod can be inserted into the catheter to stiffen the vessel section (Block 435). Both the catheter and the rod can be attached to a removable handle (Block 440). The handle can carry a tubular cutting device, or the cutting device can be introduced over the handle after the handle has been attached to the catheter and rod. An inner lumen of the cutting device provides a close sliding fit for the handle. The tubular cutting device is thus oriented coaxial with the rod and with the vessel section to be harvested (Block 445).

The cutting device is then advanced over the vessel section to core out the vessel section and tissue adjoining the vessel section (Block 450). The cutting device can be advanced by either pushing or pulling the device over the vessel section. Where the cutting device comprises two tubular members, one positioned within the other, the two tubular members can be advanced separately. For example, inner tubular member can be advanced first to hold the vessel and surrounding tissue, while outer tubular member is advanced second to cut the tissue being held by the inner tubular member. The process of incrementally advancing the inner tubular member and then the outer tubular member is repeated until the entire section has been excised. Advancing the inner tubular member ahead of the outer tubular member can protect the walls of the vessel from the cutting element positioned on the outer tubular member. Advancing and rotating the inner and outer tubular members separately can also protect the side branches of the vessel by holding them in place to achieve a clean cut at a sufficient length. The cutting device, for example, can be twisted first in one direction and then in the other direction, or it can be rotated over the vessel. The outer and inner tubular members can be twisted in opposite directions to provide a scissoring action.

The cored out vessel section and adjoining tissue are removed from the body of the patient (Block 455). Either before or after removing the vessel section and adjoining tissue, a hemostatic control method for branch vessels severed as a result of coring out the vessel section can be introduced through either the first or the second incision. The hemostatic control method can be, for example, a biological sealant, e.g., platelet gel that can be prepared from the patient's blood and injected or otherwise introduced along the track of the cutting device. The hemostatic control method can also be a thrombogenic substance such as fibrinogen, fibrin and/or thrombin placed in the track left by the cutting device. Alternatively, or in combination with a biological sealant, a biocompatible or biodegradable tube can be enclosed within the cutting device to be delivered as the cutting device is advanced over the vessel or after the cutting device has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

Hemostatic control methods are not required for embodiments of the invention as the tubular cutting device itself can exert pressure on the cut branch vessels while it remains within the patient's body. A drain can be inserted at the end of the harvesting procedure to deal with any bleeding that does occur. The site of the vessel harvesting procedure, e.g., the leg of a patient, can also be wrapped with a compression bandage to limit bleeding.

In an alternative embodiment of the invention, a rod can be inserted directly into the vessel. Thus, no guidewire and/or catheter is used. In one embodiment, a proximal portion of the vessel can be attached to the rod rather than to the catheter as described above. The handle is then attached to the rod.

In another alternative embodiment, the catheter can be inserted directly into the vessel. Thus, no guidewire or rod is used. In one embodiment, the catheter includes one or more inflatable structures, such as balloons. In yet another alternative method in accordance with embodiments of the invention, no catheter or rod is used; only a guidewire is used.

In yet another alternative embodiment, no handle is used. Instead of being carried on the handle, the cutting device is oriented coaxial with the rod. When fully inserted into the catheter within the vessel to be harvested, the rod extends far enough outside of the vessel to allow the cutting device to be aligned with the rod. The catheter can be attached to the rod before advancing the cutting device over the rod, catheter, and vessel assembly.

FIG. 2B is a flow diagram illustrating a vessel harvesting method according to another embodiment of the invention. A first incision is made at a point corresponding to a proximal end of the vessel section to be harvested (Block 405). A second incision is made at a point corresponding to a distal end of the vessel section (Block 410). A cannula is then inserted into the proximal end of the vessel section, which is located near the knee. The proximal end of the vessel is then secured to the cannula (Block 416), for example by suturing the vessel onto a barb or raised portion positioned adjacent to the distal end of the cannula. A balloon catheter is then introduced through the cannula and positioned within the vessel section (Block 421). Once positioned, the balloon is inflated to stiffen the vessel section (Block 431). A vessel-tensioning device or system is then attached to the cannula to provide a vessel-tensioning force to the vessel section (Block 436).

A cutting device is oriented coaxially with the cannula, the balloon and the vessel section to be harvested (Block 446). The cutting device is then advanced over the vessel section to core out the vessel section and tissue adjoining the vessel section (Block 450). The cutting device, for example, can be twisted first in one direction and then in the other direction, or it can be rotated over the vessel. The cored out vessel section and adjoining tissue are removed from the body of the patient (Block 455). Either before or after removing the vessel section and adjoining tissue, a hemostatic control method for treating branch vessels severed as a result of coring out the vessel section can be introduced through either the first or the second incision. The hemostatic control method can include, for example, a biological sealant, e.g., platelet gel that can be prepared from the patient's blood and injected or otherwise introduced along the track of the cutting device. The hemostatic control method can also be a thrombogenic substance such as fibrinogen, fibrin and/or thrombin placed in the track left by the cutting device. Alternatively, or in combination with a biological sealant, a biocompatible or biodegradable tube can be enclosed within the cutting device to be delivered as the cutting device is advanced over the vessel or after the cutting device has completed coring out the vessel and adjoining tissue. The tube exerts pressure on the cut branch vessels and can be either removed or, in the case of a biodegradable tube, left to dissolve or degrade over a period of a few days, for example. The space left after the removal of the vessel can also be filled with gauze to provide internal pressure to limit bleeding and absorb blood. The gauze can be removed periodically to check for absorbed blood. Limited blood collected on the gauze indicates the wound bleeding has diminished.

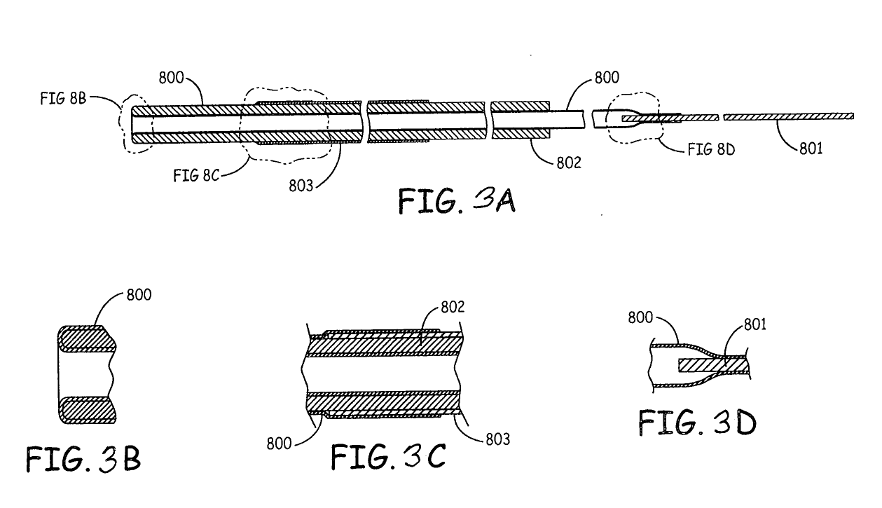

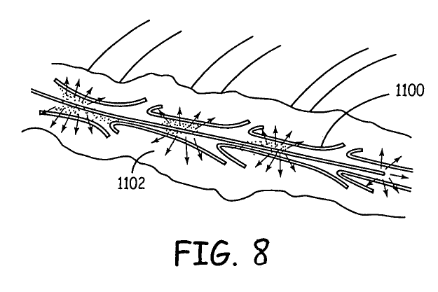

FIGS. 3A-3D illustrate a rollout intravascular sheath 800 according to one embodiment of the invention. The rollout intravascular sheath 800 can be used to introduce a stabilizing or support device, as discussed in more detail below, into a vessel while protecting the endothelial layer of the vessel. The sheath 800 can provide support to the vessel during a vessel harvesting procedure, e.g., a saphenous vein harvesting procedure. The sheath 800 can be a flexible tube, which is shown not fully extended in FIGS. 3A-3D. A rigid or semi-rigid inner tube 802 is shown advanced partially through the sliding sleeve 803. Prior to advancing the tube 802 through the sliding sleeve 803, the sheath 800 is everted around the edges of the sliding sleeve 803 and one end is fixedly attached or bonded to the sliding sleeve 803, as shown in FIG. 3C. The other end of the sheath 800 is fixedly attached or bonded to one end of a wire 801, as shown in FIG. 8D. The tube 802 is advanced over the wire 801, over a portion of the sheath 800 and through the sliding sleeve 803. The tube 802 is advanced forward into the sheath 800 and into the vessel section to be harvested. Advancement of the tube 802 causes the flexible rollout sheath 800 to unroll as it enters the vein. The wire 801 is free to move with the sheath 800 while the tube 802 is advanced forward thereby allowing the sheath 800 to be unrolled. In one embodiment, there is little to no relative motion, or sliding between sheath 800 and the interior wall of the vessel. Unrolling the sheath 800 within the vessel can minimize damage to the endothelial lining of the vessel as compared to sliding a member against the endothelial lining of the vessel as the member is advanced through the vessel.

The sheath 800 can be made of most any biocompatible material, such as polyurethane or ePTFE. In one embodiment, as the clinician advances the tube 802 in the vessel, the sheath 800 material is rolled out. While the tube 802 is advanced in the vessel, the sliding sleeve 803 is held stationary, e.g., just outside the vessel at a point adjacent the site of vessel insertion. The tube 802 is advanced in the vessel to a length that corresponds to the length of vessel that is intended to be harvested. To remove the sheath 800 from the vessel, the wire 801 is pulled back, thereby retracting the sheath 800 and the tube 802 from the vessel and, thereby creating no relative motion between the sheath 800 and the vessel.

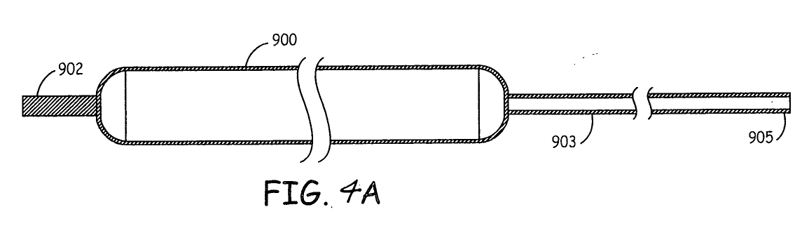

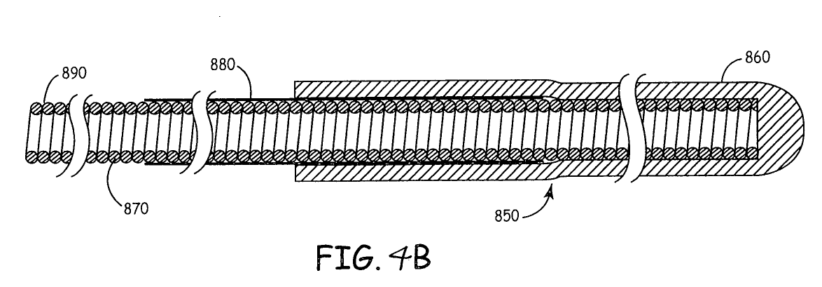

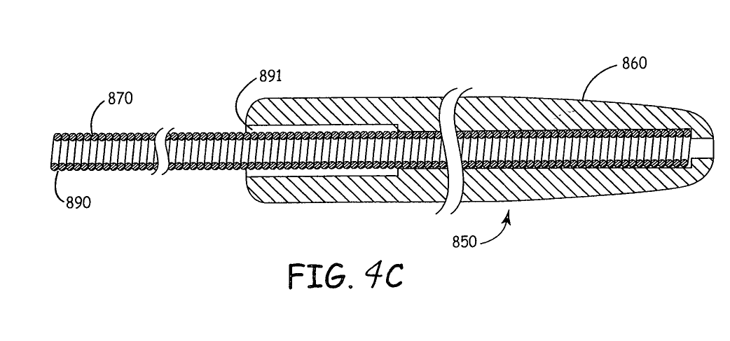

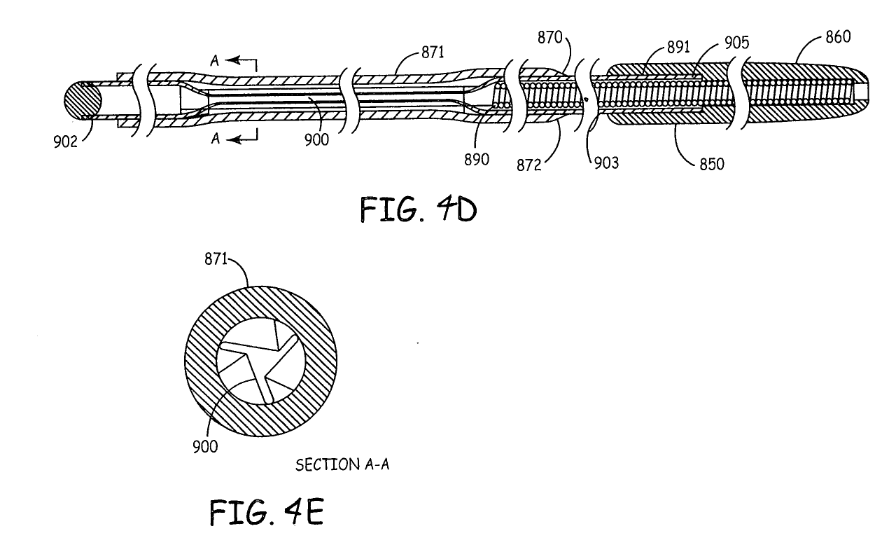

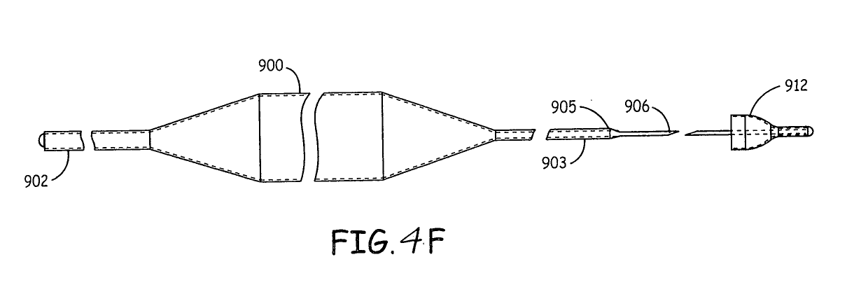

FIG. 4A illustrates a one-piece intravascular balloon catheter 900 according to one embodiment of the invention. The intravascular balloon catheter 900 is plugged at its proximal end 902 and includes extended unexpanded balloon material. In one embodiment, the balloon catheter 900 can be approximately 300-500 mm long with a 1.0-2.0 mm diameter when folded and 2.0-6.0 mm diameter when inflated. The balloon catheter 900 can be constructed of a suitable biocompatible material, such as nylon, urethane, polyethylene, or PET. The elongated distal end or routing end 903 of balloon catheter 900 is used to navigate the balloon catheter 900 through the vessel and into place.

FIG. 4B illustrates a stylet 850 that can be placed within the routing neck 903 of the balloon catheter 900 to prevent kinking of the routing neck 903 during insertion within the vessel. In one embodiment, the stylet 850 includes a flexible tip or cap 860 at its distal end, a coiled wire 870, and a membrane 880. The flexible tip 860 helps to minimize damage to the vessel wall when navigating around curves. The flexible tip 860 can be tapered to allow easy insertion into vessels of varying size. The proximal end 890 of the stylet 850 can be positioned within the routing neck 903 of the balloon catheter 900. The distal end 905 of the balloon catheter 900 is advanced over the coiled wire 870 and over the membrane 880, thereby creating a pressure fit between the distal end 905 of the routing neck 903 and the membrane 880 of the stylet 850. The pressure fit couples the stylet 850 and the balloon catheter 900 together. The coupled stylet 850 and balloon catheter 900 together can be navigated and routed through the vessel section to be harvested. In one embodiment, as shown in FIG. 4C, the stylet 850 includes a flexible tip or cap 860 at its distal end and a coiled wire 870. The proximal end 890 of the stylet 850 is positioned within the routing neck 903 of the balloon catheter 900. In this embodiment, the distal end 905 of the balloon catheter 900 is positioned within a cavity 891, thus coupling or securing the distal end 905 of the routing neck 903 and to the stylet 850, as shown in FIG. 4D.

In one embodiment, a flexible sheath 871 can be placed over the balloon catheter 900, as shown in FIGS. 4D-4E. The flexible sheath 871 can be constructed of an elastic or resilient material capable of allowing the balloon catheter 900 to be expanded or inflated and also helping to deflate or collapse the balloon catheter 900 into a low profile configuration similar to its original configuration so that the balloon catheter 900 can be easily removed from the vessel. FIG. 4E is a cross-sectional view of a deflated balloon catheter 900 within the flexible sheath 871. In one embodiment, the balloon catheter 900 is in a folded configuration when it is in a deflated or collapsed configuration within the flexible sheath 871.

FIG. 4F illustrates a flow delivered tethered balloon catheter 900 according to one embodiment of the invention. This embodiment utilizes a tether 906 coupled to the routing neck 903 to introduce the balloon catheter 900 into a vessel section, e.g., a saphenous vein, during a vessel harvesting procedure. The balloon catheter 900 is sealed at its proximal end 902. In one embodiment, the distal end 905 of the balloon catheter 900 is attached or bonded to the proximal end of the tether 906. In one embodiment, the routing neck 903 of the balloon catheter 900 can be approximately 200 mm in length. In one embodiment, the tether 906 can be approximately 500 mm in length. A parachute 912 (in one embodiment, approximately 2-5 mm in diameter) can be coupled or attached to the proximal end of the tether 906. The parachute 912 can be a cup-shaped component, a lightweight ball, or another suitable structure that is easily carried by fluid flow. The tether 906 can be a thin string, such as thread or suture material. The tether 906 can also be constructed of a material with more stiffness so that it could be pushed into the vein while injecting fluid.

FIG. 6 illustrates one embodiment of an insertion device for a flow delivered tethered balloon catheter 900, as shown in FIG. 4F. The tether 906 can be introduced into the vessel through a vessel cannula 914 connected to a Y-connector 916 with a Touhy Borst valve 918. The valve 918 can be tightened as much as possible to prevent backflow of fluid, e.g., blood or saline, but still allow the tether 906 to move. A port 920 of the Y-connector 916 is used to inject fluid, e.g., saline. The cannula 914 is inserted into the proximal end of a vessel section to be harvested, e.g., a saphenous vein section, and sutured into position. For a saphenous vein the proximal end of the section to be harvested is located near the knee. The distal end of the vessel, near the groin region, is opened to allow the parachute 912 to exit the vessel section to be harvested. The tether 906 is injected into vessel at a location near the knee using fluid, such as saline, to carry the parachute 912 from the knee to the groin incision. The balloon catheter 900 is then pulled into position within the vessel at a desired location. After the balloon catheter 900 is inflated, the cutting device is inserted at the knee incision to perform the harvest. The fluid used to advance the parachute 912 can be saline, blood, heparanized saline, or another suitable biocompatible fluid. In one embodiment, one or more fluids can be injected through the port 920 to flush the vessel before, during and/or after insertion of the balloon catheter 900. In one embodiment, the parachute 912 allows the balloon catheter 900 to be pulled into the vessel by the tether 906, rather than being pushed into the vessel with a stylet, for example.

In one embodiment, the vessel section to be harvested is isolated at its proximal and distal ends. In one embodiment, a saphenous vein section is isolated having a proximal end located approximately near the knee, while the distal end is located at or near the groin region. As shown in FIGS. 5A-5D, a distal tip 852 of a cannula 851, can be inserted into the proximal end of the isolated vessel, e.g., a section of saphenous vein. The vessel is then ligated to the cannula 851. A proximal end 853 of the cannula 851 can include a valve 854 to prevent back flow of fluid, such as blood and/or saline, from the vessel out the cannula 851 end. In one embodiment, the valve 854 is a bileaflet or duckbill valve, as shown in FIGS. 5A-5D. In one embodiment, the proximal end 853 of the cannula 851 can include a tension-coupling member 855, as shown in FIGS. 5C-5D, for coupling a tensioning member to the cannula 851. In one embodiment, a twist lock mechanism can be used to secure a tensioning device member 861, as shown in FIG. 5E, to the cannula 851. The distal end 862 of tensioning device member 861 is inserted, twisted and locked into place within tension coupling member 855 located at the proximal end 853 of the cannula 851. In one embodiment, a bayonet fastener mechanism can be used to couple the tensioning device member 861 to the tension coupling member 855. For example, raised bumps 864 sized to fit within grooves 865 can be used to couple the tensioning device member 861 to the tension coupling member 855. A tensioning device can be coupled to tensioning device member 861 at its proximal end 863.

Once the vessel is cannulated, the balloon catheter 900 can be routed through the vessel by routing the proximal neck 903 and the stylet 850 through the cannula 851 and through the vessel section to be harvested. Once the balloon catheter 900 is positioned in its desired location within the vessel section to be harvested, the stylet 850 may or may not be removed from the routing neck 903. Following placement of the balloon catheter 900 within the vessel, the balloon catheter 900 can be inflated through the distal end of the routing neck 903, which has exited out the distal end of the vessel section. In one embodiment, the balloon catheter 900 is inflated to a diameter of approximately 4 mm. The balloon catheter 900 is semi-rigid when it is inflated, which allows the vessel to still maintain most of its anatomical course. When the balloon catheter 900 is inflated, it is rigid enough to interface with the routing ridge 506, as discussed above. The routing ridge 506, in combination with a cutting device having a flexible distal end, allows the cutting device to accurately and precisely navigate the vessel to ensure the harvesting of a viable vessel section, e.g., acceptable for use as a graft in a CABG procedure.

The balloon catheter 900 can be constructed of non-compliant or semi-compliant materials, such as PET (polyethylene terepthalate), nylon, Pebax and/or polyurethane, for example. Most commonly, the balloon catheter 900 is folded and wrapped in a collapsed configuration to create a low profile to assist in its insertion into the vessel. The sheath 871 can be a section of tubing made of an elastomer such as silicone and/or modified silicone, such as C-flex, which is silicone modified styrenic thermoplastic elastomer. The sheath 871 can be applied over the top of the balloon catheter 900. The sheath 871 can expand with the balloon catheter 900 when the balloon catheter 900 is inflated with saline solution, and can return the balloon catheter 900 back to its original low profile when the balloon catheter 900 is deflated. Thus, the sheath 871 assists in an application where the balloon catheter 900 is to be inserted into a vessel with a low profile, inflated, and removed from the vessel with a low profile.

By returning the balloon to a low profile after it has been inflated inside a vessel, the amount of damage to the inner vessel walls is greatly reduced during removal of balloon catheter 900. Non-compliant and semi-compliant balloons are often folded and wrapped so that they have the lowest possible profile until they reach their destination within the vessel. Then once the balloon catheter reaches its desired area, it is inflated. Then in order to remove the balloon catheter from the vessel, the balloon catheter is deflated. However, the balloon catheter may not return to its original low profile shape when deflated. This can be destructive to the inner walls of the vessel as the balloon catheter can have edges created by folds when the balloon catheter is deflated. Therefore, the elasticity of the sheath 871 is used to bring the deflated balloon catheter 900 back to its original profile.

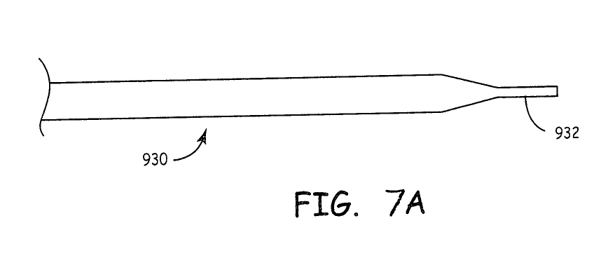

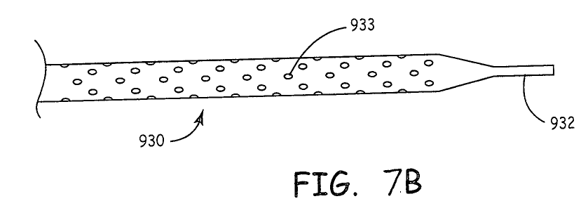

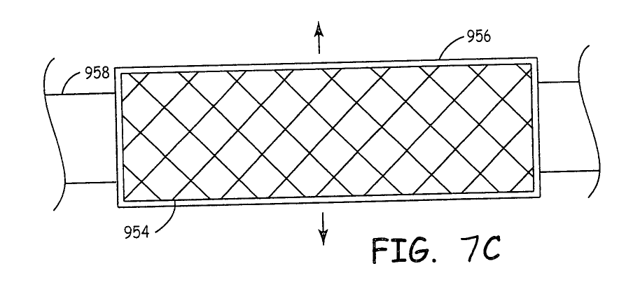

FIGS. 7A-7F illustrate vessel support devices according to various embodiments of the invention. The following discussion discloses alternatives to using the balloon concepts discussed in detail above for vessel support. Specifically, the following discussion discloses ways to provide stabilization or support to a vessel during a harvesting procedure by placing a support member inside the vessel. These alternatives include inserting a rod or dilator into a flexible sheath or coiled tube, using a wire braid that increases in diameter when compressed, a tapered rod or dilator, a rod or dilator with a flexible tip, a tube or dilator having irrigation holes, and a rod or dilator with slippery, lubricious coating, e.g., an Advawax coating or a hydrogel coating. Other lubricious coatings, as discussed above, can be used. These varied concepts all provide a support structure that is placed within the vessel section that is to be harvested, thereby providing the harvesting tool a structure to follow, while preserving the endothelial lining of the vessel. Some of the concepts provide for a small diameter during insertion and removal and a larger diameter during the cutting procedure. Some embodiments create a fluid barrier between the support member and the vessel wall.

Inserting a rod or dilator into a flexible sheath or coiled tube can be used to expand the flexible sheath or coiled tube. The flexible sheath or coiled tube can be inserted into the vessel with a smaller diameter, then expanded to a larger diameter with the rod or dilator, thereby achieving the desired diameter and stiffness. The rod or dilator can then be removed from the flexible sheath or coiled tube when it is desirable to have a smaller diameter to remove the flexible sheath or coiled tube from the vessel. The flexible sheath can be an elastomeric tube, approximately the length of the vessel section to be harvested. The flexible sheath can be capable of expanding to the desired diameter when a rod or dilator is inserted. Since the rod or dilator can be slid into the flexible sheath or coiled tube, rod or dilator and sheath materials that create minimal friction are desirable. The coiled tube can be a piece of thin-walled, coiled polymer, such as Teflon, that had a heat set in the coiled configuration. The coil can unwind as the dilator is inserted, thereby expanding to the desired diameter.

FIG. 7A illustrates one embodiment of a dilator 930 that can be placed within the vessel to be harvested. The dilator 930 has a flexible tip 932 which is narrower than the diameter of dilator 930, e.g., approximately 5 mm. The tip 932 can extend roughly 1 cm from the main body of the dilator 930 and can provide a guide for insertion of the dilator 930 into a vessel section to be harvested. The dilator 930 can be made of a Teflon material so it can slide more easily though the vessel, thereby helping preserve the endothelial lining of the vessel. In one embodiment, the dilator 930 can be inserted through a cannula 914 having diameter large enough to allow the dilator 930 to pass through. One or more fluids as discussed above can be injected through the port 920 to irrigate the vessel before, during and/or after insertion of the dilator 930.

FIG. 7B illustrates one embodiment of the dilator 930 having one or more holes 933. The holes 933 allow the user to inject one or more fluids, e.g., saline, through the dilator 930 to create pressure in the vessel thus expanding it outward and making the insertion of the dilator 930 easier. The injection of fluid can creates a fluid barrier between the dilator 930 and the vessel wall to minimize endothelial damage.

The end of the vessel can be tied off to retain the added fluid(s), such as saline. Fluid can be added to the vessel to achieve an internal vessel pressure of roughly 50-200 mmHg during insertion and removal of the dilator 930. In one embodiment, fluid(s) containing one or more medical, biological and/or pharmaceutical agents and/or drugs can be delivered to the vessel before, during and/or after a vessel harvesting procedure. One or more fluids can be delivered via one or more fluid delivery devices, e.g., a syringe or a pressurized fluid reservoir. The vessel can be secured by tying the vessel around features protruding from the side of the dilator. In one embodiment, a needle, for example, can be inserted into the vessel section to be harvested. The needle is then used to fill the vessel section with fluid(s) before, during and/or after insertion of the dilator 930. In one embodiment, a small pressure relief hole can be created in the vessel section to ensure the vessel is not damaged due to a large internal fluid pressure during the harvesting procedure. In one embodiment, a pressure gauge can be used to accurately monitor the internal pressure of the fluid filled vessel section.

FIG. 7C illustrates a vessel support device 954 including a braided cylindrical structure similar to a vascular stent. In one embodiment, a flexible protective membrane 956 is placed over the vessel support device 954 to protect the endothelial layer by shielding the vessel wall from the wire braid during insertion and removal of the vessel support device 954 during a vessel harvesting procedure. After the vessel support device 954 is inserted into the vessel, one end of the vessel support device 954 is then fixed to the vessel. An insertion tool 958 is inserted within the vessel support device 954 to cause the vessel support device 954 to expand to the diameter of the vessel.

FIG. 8 illustrates a vein illumination device according to one embodiment of the invention. As discussed, current vessel harvesting is a tedious, labor-intensive process. Harvesting is often accomplished with an electrosurgical tool to cut away tissues around the vessel to be harvested so as to free the vessel, e.g., from the leg, the chest wall or other body structure. In some harvesting procedures, the location of the vessel has to be repeatedly assessed and verified by the surgeon to be sure to stay clear of the vessel with the surgical tool to avoid damaging the vessel. To prevent bleeding from the vessel or vessel attachment points, side branches of the vessel can be occluded, for example, via clips, sutures, or electrocautery. Therefore, some embodiments of the invention include a method of illuminating the vessel from the inside out to make the location of the vessel readily visible in order to cut around it. Another embodiment involves a catheter-like device within the vessel to act as a guide for an external cutter to harvest the vessel away from the native tissue. A further embodiment controls bleeding from the vessel side branches by dispensing into the side branches a material that occludes and plugs the side branch allowing the branch to be cut without applying clips, sutures, or electrocautery. FIG. 8 illustrates illuminating a vessel 1100 with an intravenous catheter device emitting light 1102, e.g., via fiber optics. This illumination is designed to aid visualization of the vessel, e.g., the internal mammary artery (IMA), radial artery, saphenous vein or similar vasculature during cut down to aid in the vessel harvesting procedure.

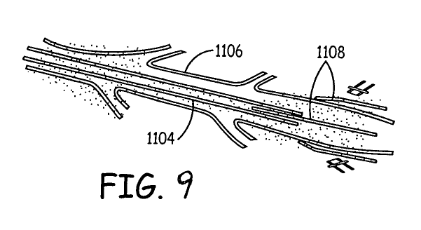

FIG. 9 illustrates an intravenous catheter device 1104 placed within a vessel 1106 to serve as a centering guide for advancing a vessel-cutting device 1108 along the exterior of vessel 1106.

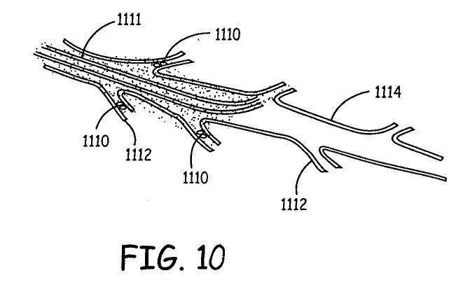

FIG. 10 illustrates a hemostatic control device 1111 according to one embodiment of the invention. Hemostatic material 1110 is shown deployed from the hemostatic control device 1111 positioned within the vessel section to be harvested. In one embodiment, vessel side branches 1112 of the vessel section to be harvested can be occluded or plugged prior to the vessel harvesting procedure. The hemostatic material 1110 can maintain hemostasis without the time consuming process of ligating or cauterizing each branch during a vessel harvesting procedure. The hemostatic material 1110 can be made of UV curable glue or adhesive, a platelet gel material, an expanding hydrogel material, and/or other biocompatible hemostatic material.

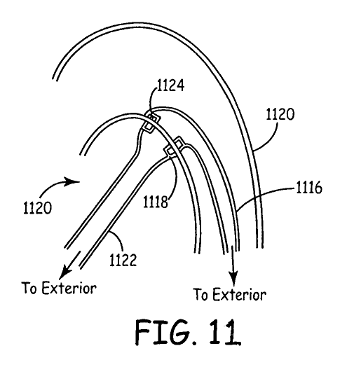

FIG. 11 illustrates a vessel location and hemostasis device according to one embodiment of the invention. In operation, a hollow guide 1122 is inserted through the chest wall, for example. A distal end of the hollow guide 1116 has a ring/oval magnet 1118 attached. The distal end is placed against vessel exterior 1120 at a target anastomosis location. The hollow guide 1116 is then placed into the vessel 1120, e.g., an IMA vessel. The hollow guide 1122 has a ring/oval magnet 1124 attached at its distal end. The intravascular hollow guide 1116 is magnetically attracted to extravascular hollow guide 1122 trapping the vessel wall between them. Once the vessel wall between the two guides is penetrated, the rings form a hemostatic seal and the hollow guides 1116 and 1122 now form a continuous channel to pass guidewires, catheters, and/or hemostatic control devices through the vessel wall.

It will be appreciated by those skilled in the art that while the invention has been described above in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

1. A system for harvesting a section of a vessel from surrounding tissue, the system comprising:
a cutting device adapted to surround the vessel along the section of the vessel and adapted to be moved along the section of the vessel in order to cut the surrounding tissue;
a catheter adapted to be inserted into the section of the vessel in order to support the vessel as the cutting device is advanced over the vessel; and
a cannula adapted to be coupled to the vessel and adapted to receive the catheter as the catheter is inserted into the section of the vessel.

2. The system of claim 1 wherein the catheter is a balloon catheter.

3. The system of claim 1 wherein the catheter is coupled to a guidewire that is inserted into the vessel before the catheter.

4. The system of claim 1 and further comprising a tensioning device coupled to the cannula to hold an end of the vessel in tension while the cutting device is advanced along the vessel.

5. The system of claim 1 wherein the catheter includes at least one lumen adapted to be coupled to at least one fluid source.

6. The system of claim 1 and further comprising a handle adapted to be removeably coupled to a proximal end of the catheter.

7. The system of claim 6 and further comprising a rod adapted to be positioned within the catheter and adapted to be attached to the handle.

8. A method of harvesting a section of a vessel from surrounding tissue, the method comprising:
making a first incision at a proximal end of the section of the vessel;
making a second incision at a distal end of the section of the vessel;
inserting a cannula into the proximal end of the vessel;
securing the proximal end of the vessel to the cannula;
inserting a catheter through the cannula and into the section of the vessel;
orienting a cutting device coaxially with the cannula and the catheter; and
advancing the cutting device over the cannula, the catheter, and the section of the vessel in order to core out the section of the vessel and a portion of the surrounding tissue.

9. The method of claim 8 and further comprising inserting a guidewire through the vessel before making the second incision.

10. The method of claim 8 and further comprising inserting a guidewire through the vessel so that a first end of the guidewire extends beyond the proximal end of the vessel and a second end of the guidewire extends beyond the distal end of the vessel.

11. The method of claim 8 wherein the catheter is a balloon catheter and further comprising inflating the balloon catheter to support the vessel as the cutting device is advanced over the vessel.

12. The method of claim 8 and further comprising attaching a tensioning device to the cannula before advancing the cutting device over the cannula.

13. An intravascular balloon catheter for use in supporting a section of a vessel being harvested from surrounding tissue with a cutting device, the catheter comprising:
a balloon including a proximal end and a distal end, the proximal end being plugged, the distal end including a routing neck, the balloon adapted to be inflated in the vessel in order to support the vessel as the cutting device is advanced along the vessel; and
a stylet coupled to the routing neck of the balloon, the stylet including a flexible tip and a coiled wire adapted to navigate through the vessel in order to position the balloon in the section of the vessel.

14. The catheter of claim 13 and further comprising a flexible sheath covering the balloon.

15. The catheter of claim 14 wherein the flexible sheath expands with the balloon when the balloon is inflated.

16. The catheter of claim 14 wherein the flexible sheath helps deflate the balloon into a low profile configuration so that the balloon can be removed from the vessel.

17. The catheter of claim 13 wherein the balloon is inflated with saline solution.

18. The catheter of claim 13 wherein the flexible tip minimizes damage to walls of the vessel.

19. The catheter of claim 13 wherein a proximal end of the stylet is positioned within the routing neck of the balloon.

20. A cannula for use in harvesting a section of a vessel, the cannula adapted to be coupled to a tensioning device, the cannula comprising:
a distal tip adapted to be inserted into and secured to a proximal end of the section of the vessel;
a valve adapted to prevent fluid flow out of the proximal end of the section of the vessel, the valve positioned in a proximal end of the cannula;
a tension-coupling member in the proximal end of the cannula, the tension-coupling member including at least one groove adapted to receive at least one raised bump of a tensioning device member adapted to be coupled to the tensioning device.

21. The cannula of claim 20 wherein the valve is one of a bileaflet valve and a duckbill valve.

22. The cannula of claim 20 wherein the distal tip includes an angled portion.

23. The cannula of claim 20 wherein a first diameter of the distal tip is smaller than a second diameter of the proximal end of the cannula.

24. The cannula of claim 20 wherein the valve is positioned between the distal tip and the tension-coupling member.

25. The cannula of claim 20 wherein the distal tip includes a ridge around which sutures can be wrapped to secure the cannula to the proximal end of the section of the vessel.
